(19) United States
(12) Patent Application Publication
(10) Pub. No.: US 2010/0273177 A1
(43) Pub. Date: Oct. 28, 2010

(54) Methods and Devices for Testing Saliva

(76) Inventors: Roger N. Piasio, Cumberland Foreside, ME (US); Nathan Turner, Portland, ME (US); Andrew Wheeler, Saco, ME (US)

Correspondence Address: FOLEY HOAG, LLP (w/ISA);PATENT GROUP, 155 SEAPORT BLVD., BOSTON, MA, 02210-2600, US

(21) Appl. No.: 12/517,774

(22) PCT Filed: Dec. 10, 2007
(86) PCT No.: PCT/US07/86987

§ 371 Date: Jul. 6, 2010

Related U.S. Application Data
(60) Provisional application No. 60/873,741, filed on Dec. 8, 2006.

(51) Int. Cl. G01N 33/53

(52) U.S. Cl. 435/7.1

(57) ABSTRACT

Provided are methods and devices for performing sensitive, rapid antigen testing of saliva, which yield sensitivity comparable to both rapid antigen tests and saliva culture.

### BACKGROUND

Throat swab culture, the current gold standard method for diagnosis of Group A Streptococcus (GAS) bacterial pharyngitis (commonly known as “strep throat”), has remained largely unchanged for over half a century. The back of the throat is swabbed with a sterile cotton swab near the tonsils. The swab is placed in a tube of culture medium, transported to a microbiology lab and streaked onto agar plates containing defibrinated blood. Plates are incubated at 37° C. for 18 to 72 hours and visually inspected for clear zones around growing bacterial colonies (beta hemolysis). As many species of bacteria that are either non pathogenic or are pathogens not associated with pharyngitis are also beta hemolytic, the presence of beta hemolytic colonies on the culture plates is not sufficient for diagnosis of GAS. Suspect colonies must be selected from the surrounding bacterial flora and sub cultured onto fresh plates until a pure culture is obtained. That isolated bacteria is tested for the presence of Lansfield Group A carbohydrate which is the definitive indicator of GAS. Throat swabbing is a relatively invasive test procedure requiring careful technique both in the initial sampling and the subsequent culture. Patients commonly experience discomfort and an unpleasant gagging sensation and results are not commonly available for several days.

A more recent alternative test method involves the use of rapid immunoassay devices for the presumptive identification of GAS in patients with pharyngitis. While these tests are much more rapid than traditional culture they are not as sensitive (75-95% sensitivity) requiring that subsequent culture be performed on all samples that test negative. Furthermore these tests also utilize a throat swab for sampling and as such are subject to the same technique dependency and invasive discomfort disadvantages as traditional culture.

As collection of saliva is a much less invasive and potentially less technique dependent procedure than swabbing the throat, and as saliva is in communication with the site of infection in pharyngitis, saliva could provide an advantageous alternative to throat swab for diagnosis. However, when saliva culture has been compared to throat swab it has been shown to be markedly less sensitive ranging from 70 to 80%^{10, 11}

Saliva poses at least two problems as a sample material that may contribute to the lack of sensitivity: (1) high viscosity within lateral flow matrices and (2) non-specific binding. Saliva is a complex matrix consisting of several components including mucopolysaccharides which can form very high molecular chains and have the ability to coat surfaces. Saliva also contains a high amount of immunoglobulin A. Because of its ability to “wash” the mouth, saliva can contain food particles and other contaminants, which along with immunoglobulin A have the potential to cause non specific binding problems.

### SUMMARY

Provided are methods and devices for performing sensitive, rapid antigen testing of saliva, which yield sensitivity comparable to both rapid antigen tests and saliva culture. The methods and devices comprise reducing non-specific binding using IgG or IgG fragments such as F(ab′)₂ as capture and/or detection antibodies, optionally coupled with enzymatic and chemical treatments to reduce viscosity. The methods and devices may be used, for example, to detect infections of Group A Streptococcus.

Further features and advantages of the present invention will become apparent from the detailed description and claims. To gain a full appreciation of the scope of the present invention, it will be further recognized that various aspects of the present invention can be combined to make desirable embodiments of the invention.

### DETAILED DESCRIPTION

Unless defined otherwise above, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

The term “analyte” refers to a substance of interest present in or absent from the sample.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The terms “comprise” and “comprising” are used in the inclusive, open sense, meaning that additional elements may be included.

The term “F(ab′)₂ IgG fragment” refers to the fragmented portion of immunoglobulin IgG which contains two antigen binding regions joined at the hinge. The fragment only contains a very small amount of the Fc region.

The term “Fc region” refers to the lower portion of immunoglobulin IgG which does not bind antigen.

The term “non-specific binding” or “NSB” refers to a generation of signal on the detection line that is not related to the presence of the analyte in the sample, i.e. a false positive.

The term “including” is used herein to mean “including but not limited to”. “Including” and “including but not limited to” are used interchangeably.

The term “saliva sample” refers to a saliva sample, from any adult or juvenile mammal.

The term “test strip” refers to a chromatographic-like medium upon which an assay may be performed.

Provided are methods for rapid antigen detection using saliva samples. Generally, the methods comprise: (a) obtaining a saliva sample, (b) preparation of the saliva sample for a detection reaction and (c) detection of pathogen antigen in the saliva sample. For example, such methods may be used to detect pharyngitis from Streptococcus Pyogenes, i.e., Group A Streptococcus.

In one embodiment, a method comprises: (a) contacting a saliva sample with a labeled IgG or IgG fragment specific for an analyte to form, if the analyte is present in the sample, a first complex comprising the labeled IgG or IgG fragment and analyte; (b) contacting the saliva sample with an IgG or IgG fragment specific for the analyte to form, if the sample comprises the first complex, a second complex comprising the first complex and IgG or IgG fragment; and (c) detecting whether the second complex is formed.

The label may include one or more of, for example, latex, dye, gold sols, radioactive, or fluorescent labels.

The analyte may be any antigen from a pathogen of interest. In certain embodiments, the analyte is a Streptococcus A antigen. For example, the antigen may be Lansfield Group A carbohydrate antigen.

In certain embodiments, the IgG or IgG fragment specific for the analyte in (b) is immobilized on a carrier. The carrier may be any suitable material for immobilizing the fragments. In certain embodiments, the carrier is a porous material, e.g. nitrocellulose.

In certain embodiments, the carrier is part of a test strip or test chamber. In some embodiments, the carrier is a test strip. In other embodiments, the carrier is part of a lateral flow device. In still other embodiments, the carrier is resin or other material suitable for packing in a column. In still other embodiments, the carrier is a plate with wells or a test tube.

In certain embodiments, the saliva sample is subjected to at least one of the following prior to the contacting: deamination, proteolysis, a surfactant and de-glycosilation. Such treatment may, for example, break down the saliva viscosity and prevent NSB, e.g., by affecting mucin associated NSB. Suitable reagents include, but are not limited to, micronitrous acid, potassium nitrosodisulfonate, bromelain, 2.5% Tween-20, 2.5% Surfonyl 440, β-Galactosidase, 2.5% Tween-20+Bromelain, 2.5% Surfonyl 440+Bromelain, Bromelain+β-Galactosidase+Tween, Mutanolysin (a N-acetylmuramidase), sodium nitrite+5% Tween 20 and 0.125N acetic acid+5% Tween 20.

In another embodiment, a method comprises (a) combining a saliva-derived sample comprising an analyte, a first IgG or IgG fragment specific for the analyte, a label, and a second IgG or IgG fragment specific for the analyte to form a complex comprising the first and second IgG and/or IgG fragments, the analyte, and the label; and (b) determining the presence of the complex. The step of combining may comprise first combining the saliva-derived sample, the first IgG or IgG fragment specific for the analyte, and the label to form a first mixture and, subsequently, combining at least some of the first mixture with the second IgG or IgG fragment.

In certain embodiments, the first IgG or IgG fragment is a labeled fragment and the label is the label of the labeled fragment.

For use in the methods described above, kits and devices for the practice of the above-described methods are also provided. Devices for practice of the methods include lateral flow devices (wherein the reagents employed in the reaction may be dried or immobilized onto a chromatographic support contained within the device), a test strip, or other support for practice of the methods. A kit for the practice of the above methods may include a support, reagents and wash and incubation buffers. Such kits and devices can contain any number or combination of reagents or components. The kits can comprise one or more of the above components in any number of separate containers, tubes, vials and the like or such components can be combined in various combinations in such containers. Kit components may be packaged for either manual or partially or wholly automated practice of the foregoing methods. Further, instructions for the use of a device or kit may be included with the device or kit. Such kits and devices may have a variety of uses, including, for example, diagnosis, therapy, and other applications.

By way of example, generally, an immunoassay device for determining the presence or amount of an analyte of interest in a sample includes a sample application member, which is in liquid communication with a conjugate pad, which is in liquid communication with a nitrocellulose test strip having a test result zone and a control zone. The immunoassay can also include a distal sink at the end opposite to the sample application pad to absorb any excess liquid after testing has run to completion.

The sample application pad is a porous pad able to absorb the sample to be tested and transfer the absorbed sample to the conjugate pad by capillary action. The conjugate pad includes one or more dried labeled molecules or reagents, such as antibodies, capable of specifically binding to the one or more analytes of interest forming a analyte-labeled reagent complex. The conjugate pad may also include one or more stabilizing compounds that are able to induce thermal stability and also stability as to conditions imposed by humidity and temperature. The conjugate pad is a porous pad able to absorb the transferred sample from the sample application pad and transfer the sample to the nitrocellulose strip by capillary action. The nitrocellulose strip is able to absorb the sample from the conjugate pad and transfer the sample by capillary action downstream to the test result zone and the control zone. The test result zone of the immunoassay device includes one or more immobilized molecules or reagents, such as antibodies or antibody fragments, capable of specifically binding to the one or more analytes of interest or any portion of the analyte-labeled reagent complex. The control zone of the immunoassay device may include one or more immobilized molecules or reagents, such as antibodies or antibody fragments, capable of specifically binding to the one or more labeled reagent.

When a liquid test sample is applied to the sample application pad of the device, the sample travels through the sample application pad, the conjugate pad, and nitrocellulose strip by capillary action. When the sample travels through the conjugate pad, the sample solublizes the dried labeled molecule or reagent, and if the analyte of interest is present in the sample, the solublized labeled molecule or reagent binds the analyte of interest forming an analyte-labeled reagent complex, otherwise, if the analyte of interest is not present in the sample, no complex is formed. The analyte-labeled reagent complex in the case of a positive test, or the labeled reagent alone in the case of a negative test, then travel to the nitrocellulose strip and travel through and pass the test result zone and the control zone of the device. If the analyte of interest is present in the sample, the analyte-labeled reagent complex binds to the immobilized reagent of the test result zone forming a detectable line, and if the analyte of interest is not present in the sample, no analyte labeled reagent complex is formed and therefore no binding occurs at the test result zone. Whether or not the analyte of interest is present in the sample to form a complex, the labeled reagent binds to the immobilized reagent of the control zone forming a detectable line indicating that the test has run to completion. Any excess liquid sample, after the testing has run to completion, can be absorbed the distal sink of the device.

In one embodiment, a device may comprise a carrier upon which is disposed (a) a sample receiving zone comprising mobilizable labeled IgG or IgG fragment specific for an analyte; and (b) a capture zone comprising immobilized IgG or IgG fragment specific for an analyte.

The analyte may be any antigen from a pathogen of interest. In certain embodiments, the analyte is a Streptococcus A antigen. For example, the antigen may be Lansfield Group A carbohydrate antigen.

In other embodiments, a device may comprise a carrier defining a flow path extending at least from a sample receiving zone to a capture zone, mobilizable labeled IgG or IgG fragment specific for an analyte disposed along the flow path, and second IgG or IgG fragment specific for the analyte disposed along the flow path; wherein: a liquid sample received by the receiving zone migrates along the flow path mobilizing the labeled IgG or IgG fragment, and in the presence of the analyte, the labeled and second IgG or IgG fragment cooperates to capture the analyte in the capture zone.

The second IgG or IgG fragment may be disposed in the capture zone when the device is in the unused state.

Optionally, reagents for reducing the viscosity of the saliva sample may comprise the sample receiving zone, capture zone or other area of the carrier. For example, such reagents as micronitrous acid, potassium nitrosodisulfonate, bromelain, 2.5% Tween-20, 2.5% Surfonyl 440, β-Galactosidase, 2.5% Tween-20+Bromelain, 2.5% Surfonyl 440+Bromelain, Bromelain+β-Galactosidase+Tween, Mutanolysin (a N-acetylmuramidase), sodium nitrite+5% Tween 20 and 0.125N acetic acid+5% Tween 20 may be dried onto the carrier. Any suitable drying methodology known in the art may be used for drying the reagents onto the carrier.

Exemplary device formats that may be used include that described in U.S. Pat. No. 6,372,513, which describes a lateral flow test having a pad with materials capable of dissociating proteinaceous materials to provide a relatively mucin-free sample.

### EXEMPLIFICATION

The invention, having been generally described, may be more readily understood by reference to the following examples, which are included merely for purposes of illustration of certain aspects and embodiments of the present invention, and are not intended to limit the invention in any way. All headings are for the convenience of the reader and should not be used to limit the meaning of the text that follows the heading, unless so specified.

### Example 1

### Use of F(ab′)2 Antibody Fragments in a Sandwich Assay Format to Reduce the Non-Specific Binding (NSB) Inherently Seen with Saliva Based Lateral Flow Based Assays

Group A Streptococcus is an organism that can be present in the mouth and causes infection in humans. It is desirable to use saliva as the sample in assays for Group A Streptococcus because it is less invasive than the conventional throat swab. An earlier test developed by Binax, Inc. for this analyte incorporated F(ab′)₂ conjugated to colloidal gold, but used whole immunoglobulin IgG for the capture line. Surprisingly, we found that when the capture line was fragmented into F(ab′)₂, the non-specific binding problem with saliva went away. Detection of the positive control was similar between the two. We collected 14 samples from both men and women and compared it to the earlier test. Without F(ab′)₂ fragments on both sides of the sandwich 6 out of 14 samples produced a nonspecific signal. With F(ab′)₂ fragments on both sides of the sandwich, 0 out of 14 samples produced a signal. All samples were cultured and none of them showed any Group A Streptococcus growth.

### Example 2

### A Novel Mechanism for the Observed FAb Fragment Mediation of Saliva NSB

It is well-known that non specific binding of antibody labels can lead to false positive results arising from the use of saliva as an immunoassay test matrix. It has been theorized that this binding results from two principal causes. The first proposed mechanism is cross-linking of capture and label antibodies via molecules that are produced by commensural bacteria (e.g. Proteins A or G of Staphylococcus Aureus) or that are present in normal human biology (e.g. rh factor). The removal of the Fc portion of capture and label antibodies has been observed to greatly reduce or even eliminate NSB associated with the saliva matrix, presumably by removing the target for the above mentioned cross-linking molecules. A second independent mechanism involves salivary mucin as NSB associated with mucin alone absent the cross-linking salivary molecules, which is observed almost universally in the immunochromatographic test (ICT) format. It has been presumed that this mucin associated NSB phenomenon results from the markedly increased viscosity of mucin solutions leading to incomplete clearance and physical entrapment of the label antibody in the capture zone of the ICT.

In an effort to better understand and ideally eliminate the mucin associated NSB phenomenon in ICT assays, a series of experiments were performed. A surrogate test for the viscosity of the sample was devised utilizing the difference in capillary rise time of solutions with different viscosities. Briefly, nitrocellulose analytical membrane (22.5 mm Millipore HF110 un-backed) was laminated to lexan by means of double sided adhesive. A wick material (⅜ inch Ahlstrom 1281) was laminated below the membrane with an approximately 2 mm overlap. 5 mm strips were cut, the wick immersed in test solution and the movement of the solution up the strip observed. The time elapsed between the initial immersion and the solution reaching the top of the strip was recorded as the capillary rise time. The effect of treatments on mucin associated NSB was determined by running samples on a group A streptococcus ICT device where the label antibody was a FAb fragment and the capture antibody contained a portion of whole antibody containing the Fc portion. A standardized artificial saliva was formulated containing a balance of physiological buffer salts similar to that of saliva, both Type I and Type II mucin in relative proportion to but in concentration approximately 2 times greater than that of saliva and cholesterol, a lipoprotein found in saliva but excluded from virtually all artificial saliva formulations. When this saliva was subjected to the capillary rise test it produced rise times substantially greater than patient saliva and when run on the ICT it produced the expected low but clearly detectable NSB.

In the attempt to reduce saliva viscosity and the attendant NSB, 4 strategies were employed: 1) deamination (removes negatively charged amino groups of mucin carbohydrate), 2) proteolysis (cleaves protein back bone of mucin into smaller subunits), 3) deglycosilation (cleaves and strips carbohydrate from mucin) and 4) surfactants (bind to mucin and may sequester molecules in micelles). The results of this testing are shown in Table 1 below.

TABLE 1

| | | Capillary | |
|---|---|---|---|
| | | Rise Time | |
| Strategy | Treatment | (sec) | ICT NSB |
| Artificial Saliva | Neat artificial saliva | 210 | +++ |
| Controls | Artificial saliva diluted 1:2 with water | 123 | ND |
| Patient saliva | C123 | 94 | ND |
| | C125 | 106 | ND |
| | C128 | 114 | ND |
| Deamination | Wet Micronitrous Acid (includes a 1:2 dilution) | 82 | − |
| | Dry Chemistry Micronitrous Acid | 120 | + |
| | Wet Potassium nitrosodisulfonate (includes a 1:2 | 93 | − |
| | dilution) | | |
| | Dry Chemistry Potassium nitrosodisulfonate | 145 | − |
| Proteolysis | Bromelain | 95 | +++ |
| Surfactant | 2.5% Tween-20 | 141 | ++ |
| | 2.5% Surfonyl 440 | 168 | ++ |
| De- | β-Galactosidase | 185 | − |
| glycosilation | | | |
| Combination | 2.5% Tween-20 + Bromelain | 83 | ++ |
| | 2.5% Surfonyl 440 + Bromelain | 95 | ++ |
| | Bromelain + B-Galactosidase + Tween 20 | 78 | − |

ND = not determined,

Three of the strategies reduced viscosity: deamination, proteolysis and, to a lesser extent, surfactants. Deglycosilation had minimal effect on viscosity. Two of the strategies, deamination and deglycosilation, either greatly reduced or eliminated the NSB associated with the artificial saliva formulation. Surprisingly, of the 2 strategies that were successful at eliminating NSB one (deglycosilation) had minimal effect on the viscosity of the sample. These results show that reducing the viscosity and hence the propensity for physical entrapment of the label is not sufficient to eliminate mucin associated NSB. However one similarity between the two strategies is clear, both modify the carbohydrate portion of the mucin molecule. This finding may further elucidate the benefit derived from immunoassays using FAb antibody fragments for saliva. The Fc portion of the antibody contains the glycosilated portion of the antibody molecule. While removal of the Fc portion of the antibody eliminates Fc specific crosslinking by Fc specific molecules it also removes carbohydrate from the capture and detection system. Since the current study implicates the unmodified carbohydrate portion of mucin, not viscosity, as the cause of mucin associated NSB, it suggests that carbohydrate mediated binding events are responsible for this NSB. Hence, the removal of the Fc portion of the antibody and with it the carbohydrate from the ICT system should be expected to eliminate the mucin associated component of the observed saliva NSB.

### Example 3

### Use of Saliva Testing Instead of Standard Throat Culture for the Detection of Streptococcus Pyogenes (Group A Streptococcus, Gas) as the Cause of Bacterial Pharyngitis

The FDA cleared BinaxNOW® StrepA test, a rapid immunochromatographic test (ICT) that detects the presence of the Lansfield Group A carbohydrate antigen from throat swabs, was modified for saliva testing.

240 subjects aged 5 to 50 years (with the majority of subjects aged 5 to 15 years) were enrolled into a study at 3 clinical site locations in the United States. Each subject provided 3 ml of saliva and 2 throat swabs collected simultaneously. One throat swab was tested immediately in the FDA cleared BinaxNOW® Strep A test and one throat swab was placed into Amies Transport Media and stored at 2-8° C. until shipped. The saliva sample was split, with 1 ml of saliva and the throat swab in transport media shipped refrigerated (2-8° C.) daily to the Streptococcal Reference Laboratory at the University of Minnesota for culture. 2 ml of saliva was shipped refrigerated (2-8° C.) daily to Binax for rapid antigen testing.

Upon receipt at Binax, saliva samples were aliquotted and frozen at −70° C. prior to rapid antigen testing. Lansfield group A antigen was extracted from any GAS present in the saliva as follows. For a 100 uL saliva sample, 1000 units of Mutanolysin (a N-acetylmuramidase), 500 uL of 2M sodium nitrite, 5% Tween 20 and 50 uL 0.125N acetic acid, 5% Tween 20 were mixed on a horizontal surface and incubated at room temperature for 15 minutes. The contents were released, incubated at room temperature for another 30 minutes.

Sample flow to the chromatographic portion of the test device was initiated and sample combined with antibody labeled colloidal gold specific for Lansfield group A carbohydrate and allowed to migrate along the chromatographic strip. Gold labeled antigen when present was captured by immobilized anti-Lansfield group A antibody forming a characteristic red line in the capture zone region of the strip. Devices were interrogated with a test reader (MaTest) that determined the intensity of signal in the capture zone. When a cutoff value of 1000 MaTest area units was used to define a positive GAS result, this marginally optimized test method yielded a sensitivity of 90%. This sensitivity compares very favorably with throat swab ICT (89%) and saliva culture (91%). While specificity was reduced to 76% compared with the gold standard throat swab culture, it not yet known to what degree this reduction in specificity is a reflection of the insensitivity anticipated for throat swab culture.^{1, 2, 3, 4} Further testing of these samples using both GAS specific real time PCR and immunoblotting techniques to clarify this potential discrepancy is currently underway.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

### REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.
1. F. R. Cockerill et al. Comparison of LightCycler PCR, Rapid Antigen Immunoassay, and Culture for Detection of Group A Streptococci from Throat Swabs: J Clin Microbiol. 2003 January; 41(1):242-9
2. Bourbeau P P, Heiter B J. Use of swabs without transport media for the Gen-Probe Group A Strep Direct Test: J Clin Microbiol. 2004 July; 42(7):3207-11.
3. Gieseker, K F, T MacKenzie, M H Roe and J K Todd. Comparison of 2 rapid Streptococcus pyogenes diagnostic tests with a rigorous culture standard. Pediatr Infect Disease J 21: 922-926, 2002.
4. Anhalt, J P, B J Heiter, D W Naumovitz and PP Bourbeau. Comparison of three methods for detection of Group A Streptococci in throat swabs. J Clin Microbiol 30: 2135-2138, 1992.
5. James, L. and Mac Farland, R. B. An epidemic of pharyngitis due to a nonhemolytic group A streptococcus at Lowry air force base. N Engl J. Med. 1971 Apr. 8; 284(14):750-2.
6. Drapkin M S, Karchmer A W, Moellering R C Jr. Bacteremic infections due to clindamycin-resistant streptococci. JAMA. 1976 Jul. 19; 236(3):263-5.
7. Taylor M B, Barkham T. Fatal case of pneumonia caused by a nonhemolytic strain of Streptococcus pyogenes. J Clin Microbiol. 2002 June; 40(6):2311-2.
8. Rubin L G, Mirkin G S. Apparent false positive detection of group a Streptococcus antigen resulting from pharyngeal infection with a nonhemolytic Streptococcus pyogenes. Pediatr Infect Dis J. 2000 July; 19(7):672-4.
9. Dierksen, K P and J R Tagg. Haemolysin-deficient variants of Streptococcus pyogenes and S. dysgalactiae subsp. Equisililis may be overlooked as aetiological agents of pharyngitis. J Med Microbiol 49: 811-816, 2000.
10. Wannamaker L. W. et al. Significance of quantitative salivary cultures for group A and non-group A and non-group A beta-hemolytic streptococci in patients with pharyngitis and in their family contacts: Pediatrics. 1979 December; 64(6):904-12.
11. Hamburger, M. 1944. Studies on the transmission of hemolytic streptococcus infections: II. Beta hemolytic streptococci in the saliva of persons with positive throat cultures. J. Infect. Dis. 75:71-78.
12. Tagg, J. R. Studies of “BLIS-ful” oral bacteria: 1991 N. Z. Dent. J. 87:14-16

1. An improved method for performing an immunoassay to detect the presence of an analyte in a saliva sample, the method comprising use of IgG and/or IgG fragments as capture and detection antibodies.

2. The method of claim 1, wherein the saliva sample is subjected to a processing step selected from the group consisting of: deamination, proteolysis, mixing with a surfactant and de-glycosilation.

3. A device, comprising a carrier upon which is disposed (a) a sample receiving zone comprising mobilizable labeled IgG or IgG fragments specific for an analyte; and (b) a capture zone comprising immobilized IgG or IgG fragments, which are also specific for the analyte.

4. The device of claim 3, wherein the analyte is a Streptococcus A antigen.

5. A method, comprising:
a. contacting a saliva sample with a first labeled IgG or IgG fragment specific for the analyte, and a second capture IgG or IgG fragment specific for the analyte; and
b. determining the presence of labeled complex as an indication of the presence of the analyte in the saliva sample.

6-7. (canceled)
